(19) United States
(12) Patent Application Publication
(10) Pub. No.: US 2026/0334877 A1
(43) Pub. Date: Oct. 29, 2026

(54) Wearable Computing Device with Improved Biometric Sensor Function

(71) Applicant: Google LLC, Mountain View, CA (US)

(72) Inventors: Michael Stephen Clarke, Seattle, WA (US); Sarah Ann Stokes Kernasovskiy, San Francisco, CA (US)

(21) Appl. No.: 19/110,732

(22) PCT Filed: Sep. 12, 2022
(86) PCT No.: PCT/US2022/043214

§ 371 Date: Mar. 11, 2025

(51) Int. Cl. A61B 5/00; A61B 5/0205; A61B 5/024; A61B 5/0531; A61B 5/332; G06F 3/01

(52) U.S. Cl. CPC A61B 5/681; A61B 5/0205; A61B 5/7221; A61B 5/742; G06F 3/015; A61B 5/02416; A61B 5/02438; A61B 5/0531; A61B 5/332

(57) ABSTRACT

A wearable computing device is provided. The wearable computing device includes a housing; a base plate coupled to the housing, the base plate defining a bottom surface of the housing, the base plate configured to contact an extremity of a user wearing the wearable computing device; a display; a biometric sensor disposed on the base plate or within the housing, the biometric sensor configured to transmit a biometric signal; and a processor. The processor is configured to obtain the biometric signal; determine a signal strength of the biometric signal does not satisfy a threshold signal strength; and responsive to determining the signal strength of the biometric signal does not satisfy the threshold signal strength, triggering a control routing comprising one or more control actions for altering the signal strength of the biometric signal. Methods for improving biometric sensor function are also provided.

### BACKGROUND

Wearable electronic devices have gained popularity among consumers. A wearable electronic device may track a user's activities or biometric data using a variety of sensors. Data captured from these sensors can be analyzed in order to provide a user with information, such as an estimation of how far they walked in a day, their heart rate, how much time they spent sleeping, and the like. However, placement of the wearable computing device on the user's body affects the signal quality obtained from the biometric sensor(s), which can in turn affect the accuracy of any biometrics determined based on the signals.

### SUMMARY

Example aspects of the present disclosure are directed to a wearable computing device capable of facilitating, or independently executing, a control routine (e.g., a closed-loop (feedback) control routine) to optimize biometric sensor functioning. In some implementations, the wearable computing device is capable of facilitating the user's placement (e.g., adjustment of tightness and/or other variables of placement relative to the limb, such as degree of rotation or distance along the proximal-distal axis) of the wearable computing device on their extremity to ensure the signal strength obtained from the biometric sensor(s) satisfies a threshold signal strength that is needed to accurately measure biometrics of the user. In some implementations, the wearable computing device (e.g., using a processor of the wearable computing device) can be configured to compare a signal strength from the biometric sensor(s) to the threshold signal strength to determine whether the signal strength satisfies a threshold signal strength. When it is determined that the signal(s) obtained from the biometric sensor do not satisfy the threshold signal strength, one or more control actions can be triggered for altering the signal strength.

In some implementations, a notification may be output prompting the user to modify placement of the wearable computing device on the user's extremity (e.g., to modify position, tightness and/or orientation of the wearable computing device on the user's extremity).

In other implementations, if the threshold signal strength is not satisfied, one or more actuators on the wearable computing device may be utilized to self-adjust placement of the wearable computing device (e.g., extend or retract the base plate relative to the rest of the housing, which may also result in adjusting an outer contour of the wearable computing device) such that threshold signal strength can be achieved. The one or more control actions may thus comprise utilizing one or more actuators to adjust the base plate against the extremity of the user until at least one placement criterion is fulfilled. Accordingly, the wearable computing device may be configured to detect fulfillment of at least one placement criterion for controlling, and in particular stopping an adjustment of the base plate. A placement criterion may for example include placement of the wearable computing device within a placement range pre-determined as optimal for the biometric signal satisfying the threshold signal strength. Generally, one or more actuators may be utilized in addition to or instead of notifying the user to manually modify placement of the wearable computing device. In some implementations, a corresponding notification may be output first and the one or more actuators may be subsequently utilized in response to a user input or elapsing of a timer without the user succeeding in achieving a proper placement.

In some implementations, the wearable computing device can include one or more bands that can be adjusted (e.g., tightened or loosened) to achieve proper placement of the wearable computing device on the extremity of the user, wherein “proper” placement in the present context relates to a placement of the wearable computing device resulting in the biometric signal satisfying the threshold signal value. Accordingly, the wearable computing device may comprise one or more bands configured to attach the wearable computing device to the extremity of the user and being (manually and/or automatically) adjustable in response to the one or more control actions. For example, the bands can be tightened around the wrist of the user and held in place using a spring-loaded clasp instead of the more common tang buckle. Such a clasp allows for tightness of the bands to be optimized more precisely, in a continuous, fine-grained way in comparison to the limited number of discrete holes for the tang on a conventional watch band, thus allowing the bands to be more securely fastened to the extremity and reducing movement of the device on or around the extremity. For instance, while the clasp's spring-loaded button is depressed by the user so that it in the “open” position, the outer two ends of the bands can be threaded through the clasp and pulled tight against the user's extremity until the device indicates that the threshold signal strength is satisfied, at which point the user can release the clasp's button and the clasp will prevent the bands from sliding. Additionally, the end attachments of the bands can include an elastic material such that a small degree of over-tightening or under-tightening of the bands would be partially counteracted, so that the overall tightness stays within a desired target window (between the lower threshold of tightness, i.e. being too loose for good signal quality, and the upper threshold of tightness, after which signal quality and/or comfort starts to decline significantly).

The wearable computing device according to example embodiments of the present disclosure can provide numerous technical effects and benefits. For instance, detection and display of a notification indicating that the threshold signal strength is not satisfied, allows the user, or the device itself, to adjust placement of the device on the extremity of the user to ensure proper functioning of all biometric circuitry disposed on the device. In this manner, biometric markers determined based, at least in part, on signals obtained from the biometric sensors can be improved. Furthermore, since the user or the device's processor determines proper placement of the wearable computing device on the user's extremity based on a signal quality of signals obtained from the biometric sensors, no additional hardware (e.g., position or pressure sensors) is needed. Accordingly, in view of the form factor for wearable computing devices, configurations of the device eliminate the need for additional sensors or components. Further, band embodiments disclosed herein offer more continuous, fine-grained control over tightness as compared to other prong and buckle bands.

### BRIEF DESCRIPTION OF THE DRAWINGS

Various embodiments in accordance with the present disclosure will be described with reference to the drawings, in which:

FIG. 1 depicts a wearable computing device shown on the extremity of a user according to some implementations of the present disclosure.

FIG. 2 depicts a side view of the housing of a wearable computing device according to some implementations of the present disclosure.

FIG. 3 depicts a top-down view of the base plate according to some implementations of the present disclosure.

FIG. 4 depicts a side view of the housing of a wearable computing device according to some implementations of the present disclosure.

FIG. 5A depicts a side view of the housing of a wearable computing device according to some implementations of the present disclosure.

FIG. 5B depicts a side view of the housing of a wearable computing device according to some implementations of the present disclosure.

FIG. 5C depicts a side view of the housing of a wearable computing device according to some implementations of the present disclosure.

FIG. 6 depicts a set of basic components of a wearable computing device according to some implementations of the present disclosure.

FIG. 7 depicts a display of the wearable computing device according to some implementations of the present disclosure.

FIG. 8 depicts a wearable computing device shown on the extremity of a user according to some implementations of the present disclosure.

FIG. 9 depicts a wearable computing device according to some implementations of the present disclosure.

FIG. 10 depicts a wearable computing device according to some implementations of the present disclosure.

FIG. 11 depicts a flow diagram of an example process for improving biometric sensor function of the device according to some implementations of the present disclosure.

FIG. 12 depicts another flow diagram of an example process for improving biometric sensor function of the device according to some implementations of the present disclosure.

FIG. 13 depicts yet another flow diagram of an example process for improving biometric sensor function of the device according to some implementations of the present disclosure.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Systems and methods in accordance with various embodiments of the present disclosure may overcome one or more of the aforementioned and other deficiencies experienced in conventional approaches for wearable computing devices, such as electronic wellness trackers. For example, in various embodiments, capabilities may be integrated into the wearable computing device to enable the device to attach more securely to the user's extremity, such that a threshold signal quality for biometric sensors can be satisfied, while at the same time avoiding over tightening that could cause discomfort and other negative effects.

Wearable computing devices must be properly affixed to the user's body to ensure proper function of biometric sensors. Many users may wear the device too loose, which can reduce the biometric sensor's ability to achieve a continuous and good quality signal from the user. For certain wrist worn electrodermal activity (EDA) sensors, sufficient and continuous contact between the user's skin and electrodes disposed on the base plate of the device is necessary in order to minimize contact impedance as well as intermittent interruptions of electrical continuity, and thus achieve proper signal quality for the EDA sensor. Similarly, wrist worn electrocardiogram (ECG) sensors utilize electrodes disposed on the base plate of the device that require contact from the user's skin, as well as two electrodes on the sides or top bezel of the device that require contact from the fingers of the user's other hand, in order to generate a signal. Oftentimes, unbeknownst to the user, the device is worn in such a manner that there is not sufficient contact between components of the device and the user's skin. It has been estimated that wearable computing devices can be out of contact with the user's skin up to 10% or more of wear time, which can negatively affect the accuracy and function of the EDA or ECG sensor. Accordingly, for both EDA and ECG sensors sufficient contact between the wearable computing device and the user's skin is necessary for the biometric sensor to obtain a sufficient signal.

Certain other biometric sensors (e.g., photoplethysmography (PPG) sensors) utilize emitted light in order to obtain a signal from the user. For example, light is emitted onto the user's skin. At least a portion of the emitted light is absorbed within the skin depending on certain variables (e.g., blood oxygen saturation), while other portions of the emitted light are reflected back to the sensor. This change, calculated as the difference between the amount emitted versus the amount reflected back, is utilized by the PPG sensor to determine biometrics of the user. Thus, the higher the fraction of emitted light from the PPG sensor that enters the wrist and is reflected back, the stronger the signal. Thus, when the device is worn too loose, emitted light may not be transferred effectively into the user's skin resulting in a weak signal. However, when the device is worn too tight against the user's skin, blood can be forced out of the capillaries underneath the wearable computing device which can blanch the skin causing a decrease in captured reflected light resulting in decreased signal strength. Accordingly, proper placement on the user's wrist is also important for emitted light biometric sensors as well.

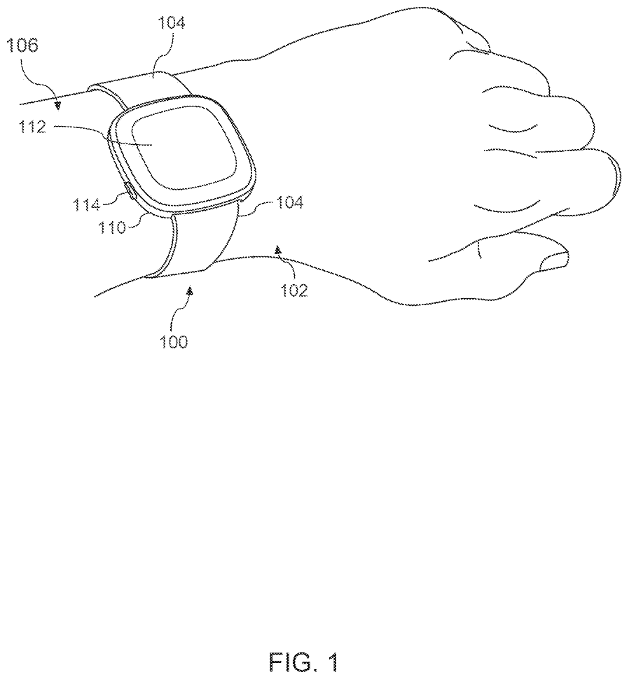

FIG. 1 depicts a wearable computing device 100 according to some implementations of the present disclosure. As shown, the wearable computing device 100 can be worn, for instance, on an extremity 106 such as a wrist 102 of a user. For instance, the wearable computing device 100 can include one or more bands 104 and a housing 110. The housing 110 can be coupled to the band 104. In this manner, the band 104 can be fastened to the wrist 102 of the user to secure the housing 110 to the wrist 102 of the user. The one or more bands 104 can be configured to attach the wearable computing device 100 to the extremity 106 of the user. The one or more bands 104 are capable of being manually and/or automatically adjustable in response to the one or more control actions, as described further hereinbelow.

In some implementations, the wearable computing device 100 can include a display 112 that can display content (e.g., time, date, etc.) to the user. In some implementations, the display 112 can include an interactive display (e.g., touchscreen or touch-free). In such implementations, the user can interact with the wearable computing device 100 via the display 112 to control operation of the wearable computing device 100. Alternatively, or additionally, the wearable computing device 100 can include one or more user inputs 114 that can be manipulated by the user to interact with the wearable computing device 100. For instance, the one or more user inputs 114 can include a mechanical button that can be manipulated (e.g., pressed) to interact with the wearable computing device 100. In some implementations, the one or more user inputs 114 can be manipulated to control operation of a backlight (not shown) associated with the display 112. It should be understood that one or more user inputs 114 can be configured to allow the user to interact with the wearable computing device 100 in any suitable manner. For instance, in some implementations, the one or more user inputs 114 can be manipulated by the user to navigate through one or more menus on the display 112.

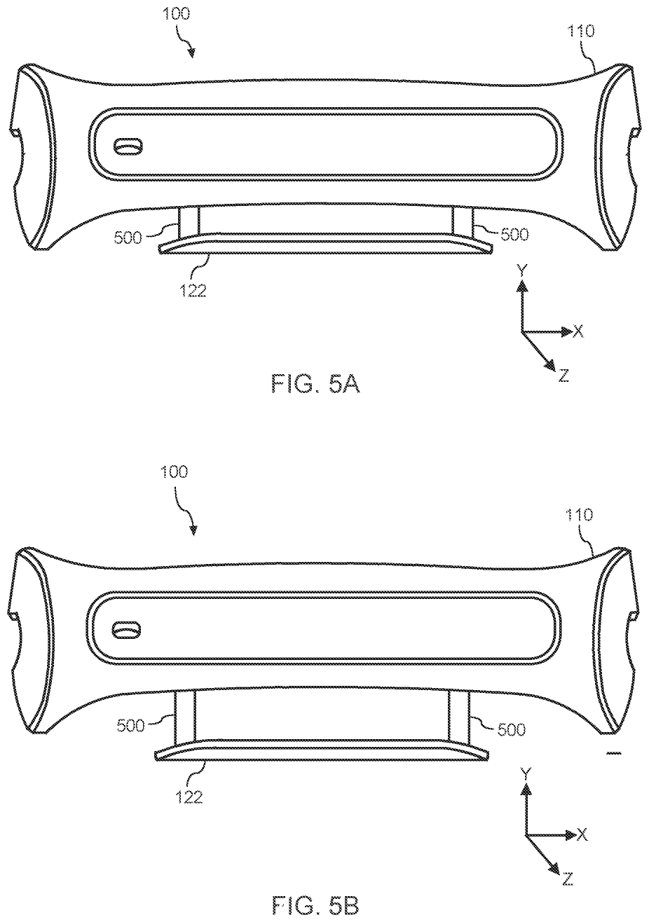

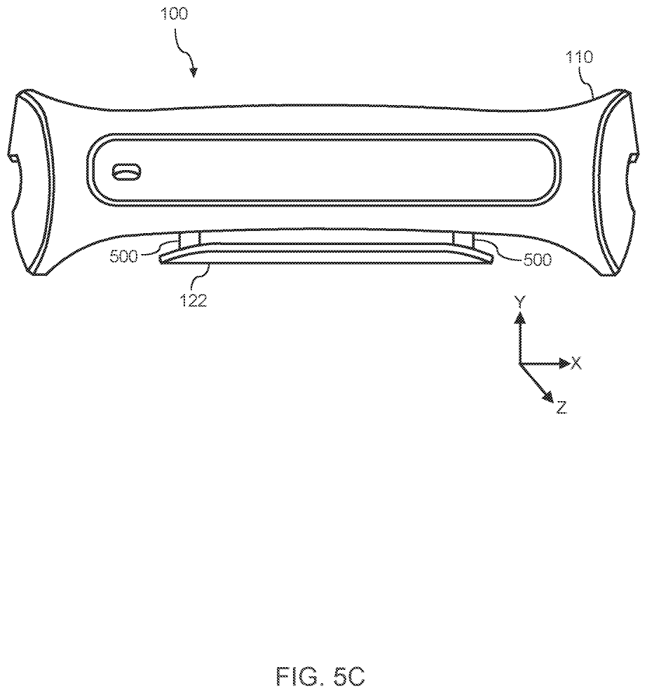

The user input 114 can also include a crown that can be rotated to allow the user to scroll through options on the display 112. Further, the user input 114 can be pulled away from the housing 110 to a second position in order to further access or engage internal features of the wearable computing device 100. Whilst in the second position, the user input 114 can also be rotated. The user input 114 can also be configured to engage one or more racks and/or pinions disposed within the housing 110. For instance, as will be discussed further hereinbelow with reference to FIGS. 5A-5C, the user input 114 can be coupled to one or more actuators 500 in order to move the base plate 122 with respect to the housing 110. Mechanical components used in timepieces, such as mechanical watches, including known racks and pinions, can be incorporated into the wearable computing device 100 as necessary.

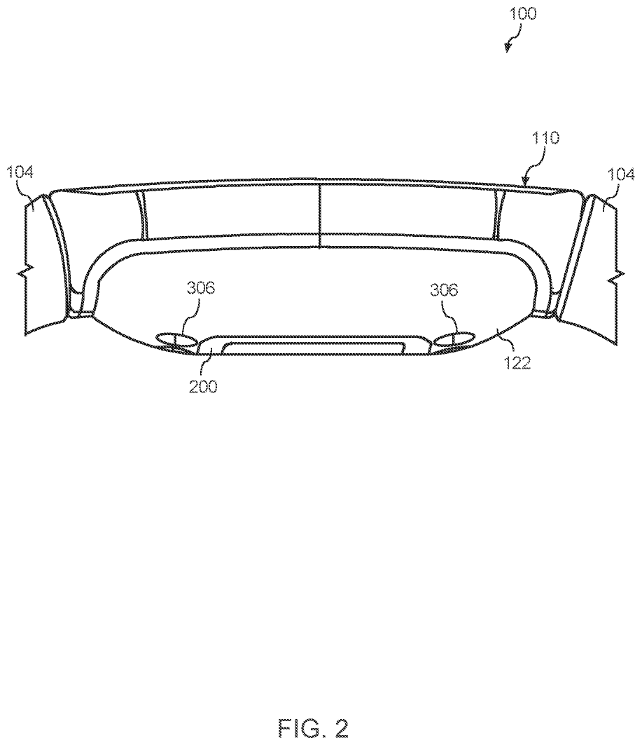

The housing 110 may be a multi-part component, such that the housing 110 is split into a first part and a second part. However, it should be appreciated that there may be additional parts. Moreover, in embodiments, additional components may be utilized to form one or more parts. For example, the base plate 122 may form a portion of the housing 110 (shown in FIG. 2). The housing 110 may enclose one or more electronic components, which may be utilized to collect and/or analyze data, as described herein. For example, the housing 110 may enclose appropriate circuitry for biometric sensors, such as ECG, PPG, and/or EDA measurements. Additionally, the housing 110 can enclose appropriate circuitry for other sensors, such as optical flow sensors, that can be utilized according to the present disclosure.

In some implementations, the wearable computing device 100 can be designed to be worn (e.g., continuously) by the user. When worn, the wearable computing device 100 can gather data regarding activities performed by the user, or regarding the user's physiological state. Such data may include data representative of the ambient environment around the user or the user's interaction with the environment. For example, the data can include motion data regarding the user's movements, ambient light, ambient noise, air quality, etc., and/or physiological data obtained by measuring various physiological characteristics of the user, such as heart rate, perspiration levels, body temperature, and the like. The wearable computing device 100 may also be referred to as a wearable or a fitness tracker, and may also include devices that are worn around the chest, legs, head, or other body part, or a device to be clipped or otherwise attached onto an article of clothing worn by the user.

As shown, the wearable computing device 100 positioned on the wrist 102 of an extremity 106 of the user, which is the user's left arm. During operation, the user may swing their extremity 106 while walking, or change position of their extremity 106 for a variety of reasons, and as a result it may alter the placement of the wearable computing device 100 on the skin of the user's extremity 106 (e.g., their arm). When such displacement of the wearable computing device 100 happens, it may be difficult to obtain proper signals for various biometric sensors disposed on the wearable computing device 100, such as ECG, EDA, PPG, or other biometric sensors.

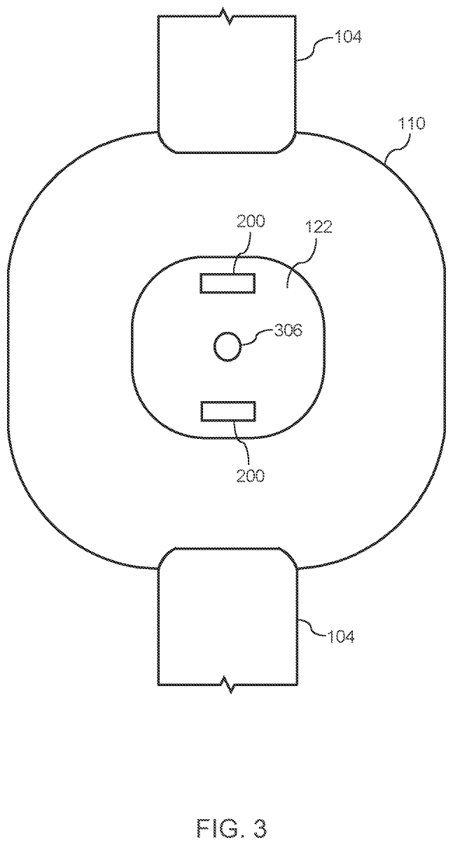

Now referring to FIGS. 2-3, in embodiments, the wearable computing device 100 may include a base plate 122 configured to be positioned against the extremity 106 of the user as illustrated in FIG. 1. The base plate 122 is configured along the bottom side of the housing 110. This base plate 122 can include one or more components for the biometric sensors. As shown, the base plate 122 includes an ECG electrode 200. As shown, the ECG electrode 200 can be positioned within an opening (e.g., cutout) defined by the base plate 122. In this manner, the ECG electrode 200 can be positioned against the wrist 102 (FIG. 1) of the user when the housing 110 is secured to the wrist 102 of the user via the band 104. When the ECG electrode 200 is in contact with the wrist 102 of the user, the ECG electrode 200 can be electrically connected to the wrist 102 of the user. Furthermore, it should be understood that the wearable computing device 100 can determine one or more health metrics (e.g., heart rate) of the user based, at least in part, on data obtained via the ECG electrode 200 when the ECG electrode 200 is electrically connected to the wrist 102 of the user.

The ECG electrode 200 can be connected to an ECG circuit that can detect small changes in electrical charge on the skin that vary with the user's heartbeat. ECG signals can be monitored over time to attempt to determine irregularities in heartbeat that might indicate serious cardiac issues. ECG measurements are obtained by measuring the electrical potential of the heart over a period of time, typically corresponding to multiple cardiac cycles. By a user wearing the device having the ECG electrode 200 disposed thereon against their skin for a minimum period of time, during which ECG measurements are taken, the wearable computing device 100 can collect and analyze the ECG signal and provide feedback to the user.

Embodiments of the present disclosure may include a system that includes at least two independent electrodes, electrically isolated within a single device. For example, at least two electrically isolated ECG electrodes can be disposed on the base plate 122 of the wearable computing device 100 (not shown in FIG. 2). The base plate 122, or a portion thereof, may contact the wrist 102. As will be appreciated, the base plate 122 may have one of the largest continuous surface areas for the wearable computing device 100, thereby achieving a goal described above to increase surface area and reduce contact impedance between the skin and the electrodes. In various embodiments, a first electrode is formed from a conductive electrode material and may be electrically isolated from the remainder of the device and from a second electrode, for example by incorporating insulating material into the wearable computing device 100, such as plastics and the like. A second electrode can also be disposed on the base plate 122 or may be disposed on another area of the wearable computing device 100, such as on a portion of the outer surface of the housing 110. It should be appreciated that the second electrode may further comprise two separate, electrically isolated electrodes.

An optical PPG sensor, including an emitter and detector, is configured to the wearable computing device 100. As previously noted, PPG signals can be recorded using a light source (e.g., an LED) and a corresponding light detector (e.g., a photodiode). With each cardiac cycle the heart pumps blood to the periphery. Even though this pressure pulse is somewhat damped by the time it reaches the skin, it is enough to distend the arteries and arterioles in the subcutaneous tissue. The change in volume caused by the pressure pulse is detected by illuminating the skin with the light from a light-emitting diode (LED) and then measuring the amount of light either transmitted or reflected to a photodiode.

Where optical sensors are disposed or arranged on the skin-side of the wearable computing device 100, in operation, a light source in the wearable computing device 100 may emit light upon the skin of the user and, in response, a light detector in the wearable computing device may sample, acquire, and/or detect corresponding reflected and/or emitted light from the skin (and from inside the body). The one or more light sources and light detectors may be arranged in an array or pattern that enhances or optimizes the signal-to-noise ratio and/or serves to reduce or minimize power consumption by the light sources and light detectors. These optical sensors may sample, acquire and/or detect physiological data which may then be processed or analyzed (for example, by resident processing circuitry) to obtain data that is representative of, for example, a user's heart rate, respiration, heart rate variability, oxygen saturation (SpO2), blood volume, blood glucose, skin moisture, and/or skin pigmentation level.

The light source(s) may emit light having one or more wavelengths that are specific or directed to a type of physiological data to be collected. Similarly, the optical detectors may sample, measure and/or detect one or more wavelengths that are also specific or directed to a type of physiological data to be collected and/or a physiological parameter (of the user) to be assessed or determined. For instance, in one embodiment, a light source emitting light having a wavelength in the green spectrum (for example, an LED that emits light having wavelengths corresponding to the green spectrum) and a photodiode positioned to sample, measure, and/or detect a response or reflection corresponding with such light may provide data that may be used to determine or detect heart rate. In contrast, a light source emitting light having a wavelength in the red spectrum (for example, an LED that emits light having wavelengths corresponding to the red spectrum) and a light source emitting light having a wavelength in the infrared spectrum (for example, an LED that emits light having wavelengths corresponding to the IR spectrum) and photodiode positioned to sample, measure and/or detect a response or reflection of such light may provide data used to determine or detect SpO2.

Indeed, in some embodiments, the color or wavelength of the light emitted by the light source, e.g., an LED (or set of LEDs), may be modified, adjusted, and/or controlled in accordance with a predetermined type of physiological data being acquired or conditions of operation. Here, the wavelength of the light emitted by the light source may be adjusted and/or controlled to optimize and/or enhance the “quality” of the physiological data obtained and/or sampled by the detector. For example, the color of the light emitted by the LED may be switched from infrared to green when the user's skin temperature or the ambient temperature is cool in order to enhance the signal corresponding to cardiac activity.

The wearable computing device 100, in some embodiments, may include a window 306 (for example, a window that is, to casual inspection, opaque) in the housing 110 or the base plate 122 to facilitate optical transmission between the optical sensors (e.g., the emitter and detector) and the user. Here, the window 306 may permit light (for example, of a selected wavelength) to be emitted by, for example, one or more LEDs, onto the skin of the user and a response or reflection of that light to pass back through the window 306 to be sampled, measured, and/or detected by, for example, one or more photodiodes. In one embodiment, the circuitry related to emitting and receiving light may be disposed in the interior of housing 110 of the wearable computing device 100 and underneath or behind a plastic or glass layer (for example, painted with infrared ink) or an infrared lens or filter that permits infrared light to pass but not light in the human visual spectrum. In this way, the light transmissivity of window 306 may be invisible to the human eye.

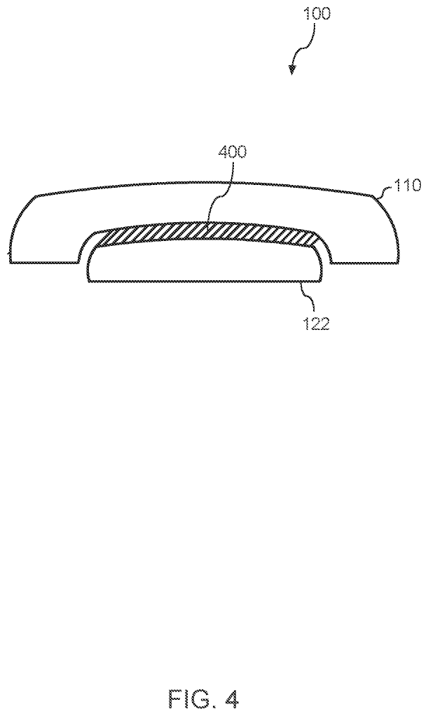

Referring now to FIG. 4, in certain embodiments, the base plate 122 including certain components for the biometric sensors is spring-mounted to the housing 110. For instance, the base plate 122 can be coupled to the housing 110 via an elastic material 400. Disposition of the elastic material between the base plate 122 and the housing 110 can facilitate more continuous attachment of the base plate 122 against the user's skin during use of the wearable computing device 100. For instance, when the user initially attaches the housing to their wrist with the appropriate amount of pressure, the elastic material will become slightly compressed. When the user subsequently engages in vigorous movement of their arm (e.g., during exercise), the inertia of the wearable computing device 100 causes the housing 110 to begin to move slightly farther away from the wrist to the amount that the band 104 will accommodate. As this movement of the housing 110 away from the wrist occurs, the elastic material 400 can expand back towards its uncompressed height, thereby offsetting the movement of the housing to ensure that the base plate 122 remains in sufficient contact with the user's extremity to maintain good signal quality. Similarly, should the wearable computing device 100 be placed on the user's extremity in such a manner that it would be too tight against the skin (e.g., by the loop of the wristband being made too short), the elastic material can further compress to partially counteract this over-tightening such that the base plate 122 is not disposed with too much pressure against the user's extremity. Accordingly, the elastic material 400 can provide expansion or contraction depending on whether or not the wearable computing device 100 is being worn in a manner that is too tight or too loose. Accordingly, the elastic material 400 can, within a certain tolerance range, improve attachment of the device and more specifically the base plate 122 on the skin of the user. Suitable elastic materials can include elastomers, rubbers, nylons, thermoplastics, vinyls, or combinations thereof.

As illustrated in FIG. 5A, the base plate 122 can be coupled to the housing 110 utilizing one or more actuators 500. Without being bound by any particular theory, the actuator 500 can include any type of actuator, such as electric, hydraulic, pneumatic, mechanical, etc. The actuator 500 is capable of converting energy received into mechanical movement. In certain embodiments, the actuator 500 is configured to convert electrical energy into mechanical energy to move the base plate 122 with respect to the housing 110. In other embodiments, however, the actuator 500 can be configured to convert rotary motion into linear motion. In certain embodiments, a motor (not shown) disposed in the housing 110 can be configured to translate the actuators 500. In other embodiments, the actuators 500 can include a voice coil.

As illustrated in FIG. 5B, the actuators 500 can move the base plate 122 further away from the housing 110 with respect to the Y-direction. Such movement of the base plate 122 by the actuators 500 as shown in FIG. 5B can be used to provide better placement of the base plate 122 against the skin of the user to improve biometric signals as will be discussed further hereinbelow. For instance, such movement of the actuators 500 in accordance with FIG. 5B can be used to adjust contact of the base plate against the extremity of the user until a threshold signal strength is achieved. Movement of the base plate 122 by actuators 500 as shown in FIG. 5B can be used to compensate for loose placement of the wearable computing device 100 against the skin of the user. In other embodiments, such as those illustrated in FIG. 5C, the actuators 500 can be used to move the base plate 122 in an opposite direction with respect to the movement of FIG. 5B, such as back towards the housing 110 in the Y-direction. Movement of the base plate 122 by actuators 500 in accordance with FIG. 5C can be used to compensate for over-tight placement of the wearable computing device 100 against the skin of the user.

As noted, the actuators 500 can be configured to facilitate converting rotary motion into a linear motion. For example, the user input 114 can be a crown that can be rotated. In order to move the actuators 500 in and out with respect to the Y-direction, the user can rotate the user input 114 in a first direction to move the actuators 500 in a first direction (e.g., movement of the base plate 122 away from the housing 110) and the user can rotate the user input 114 in a second direction to move the actuators 500 in a second direction (e.g., movement of the base plate 122 in towards the housing 110). In such embodiments, the actuators 500 are configured to convert mechanical rotary motion from the user input 114 into mechanical linear motion.

Further, it should be noted that while general Y-direction movement of the base plate 122 by the actuators 500 is provided, the disclosure is not so limited. Indeed, any number of actuators 500 can be utilized to couple the base plate 122 to the housing 110. In such embodiments, the actuators 500 can each be individually controlled such that portions of the base plate 122 extend farther in the Y-direction as compared to other portions of the base plate 122. In such embodiments, the actuators 500 can adjust placement of the base plate 122 across uneven surfaces of the user's extremity. For example, should the wearable computing device 100 be placed over a portion of the user's wrist bone, the actuators 500 can modify placement of the base plate 122, to compensate for protrusions against the base plate 122 due to the user's wrist bone. Indeed, any number of actuators and placement of actuators can be utilized as disclosed herein. Additionally, while linear actuators are illustrated, the disclosure is not so limited and actuators capable of moving the base plate in a variety of directions with respect to the X-axis or Z-axis are also contemplated.

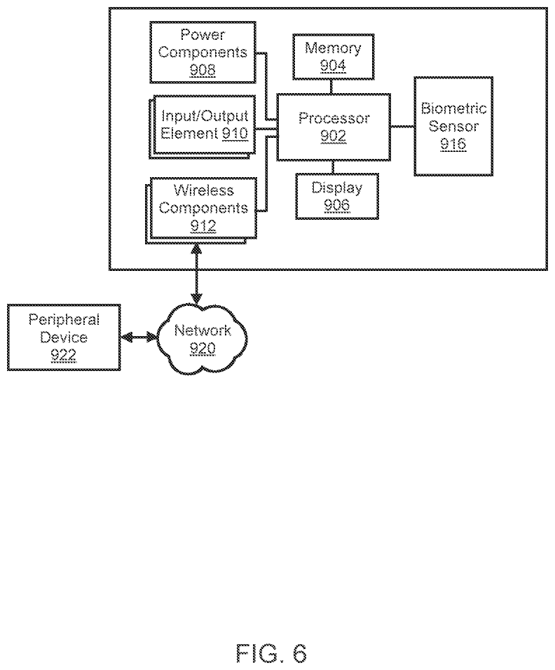

The actuators 500 can be operated in a variety of manners. For instance, in certain embodiments the actuators 500 can be operated from input provided by the user. For example, the user can access a software application (e.g., mobile app) stored in memory of the wearable computing device 100 to operate the actuators 500. For instance, in some implementations, the user can interact (e.g., touch) with the display 112 to open the software application and provide instructions for controlling operation of the actuators 500. Still, in other embodiments, the user may utilize the one or more user inputs 114 (e.g., a mechanical button or dial) to operate the actuators 500. Further, as will be discussed further hereinbelow, the wearable computing device 100 can include a processor 902 (FIG. 6) configured to operate the actuators 500. For example, the processor 902 can be configured to operate the actuators 500 based, at least in part, on signals received from the one or more biometric sensors.

FIG. 6 illustrates a set of basic components 900 of one or more devices of the present disclosure, in accordance with various embodiments of the present disclosure. In this example, the device includes at least one processor 902 for executing instructions that can be stored on the memory 904. As would be apparent to one of ordinary skill in the art, the device can include many types of memory, data storage or computer-readable media, such as a first data storage for program instructions for execution by the at least one processor 902, the same or separate storage can be used for images or data, a removable memory can be available for sharing information with other devices, and any number of communication approaches can be available for sharing with other devices. The device also includes one or more power components 908, such as a battery, including a rechargeable battery. The device may include at least one type of display 906, such as a touch screen, electronic ink (e-ink), organic light emitting diode (OLED) or liquid crystal display (LCD), although devices such as servers might convey information via other means, such as through a system of lights and data transmissions. The device typically will include one or more wireless components 912, such as a port, network interface card, or wireless transceiver that enables communication over at least one network 920. The device can also include at least one input device 910 able to receive input from a user. This input can include, for example, a push button, touch pad, touch screen, wheel, joystick, keyboard, mouse, trackball, keypad or any other such device or element whereby a user can input a command to the device. In certain embodiments, the input device 910 can include the display 906. While implementations are disclosed with reference to a processor, in some implementations an application specific integrated circuit can be utilized instead of or in addition to a processor.

The device can be wirelessly connected to one or more peripheral devices 922. Examples of such peripheral devices 922 include personal computers, cell phones, handheld messaging devices, laptop computers, set-top boxes, personal data assistants, electronic book readers and the like. The wireless components 912 on the device can be wirelessly connected to the peripheral device 922 via the network 920. Protocols and components for communicating via such a network 920 are well known and will not be discussed herein in detail. Communication over the network 920 can be enabled via wired or wireless connections and combinations thereof. In this example, the network includes the Internet, as the environment includes a Web server for receiving requests and serving content in response thereto, although for other networks, an alternative device serving a similar purpose could be used, as would be apparent to one of ordinary skill in the art.

The device includes one or more biometric sensors 916. The biometric sensor 916 can include any of an ECG, PPG, EDA, any bioimpedance sensor, and combinations thereof. For instance, the biometric sensor 916 can include both an ECG sensor and a PPG sensor. Notably, the biometric sensor 916 can include biometric sensing components that are electrically coupled to biometric sensor circuitry disposed within the housing of the device. For instance, in embodiments where the biometric sensor 916 is an ECG sensor, the device includes or more electrodes electrically coupled to biometric circuitry. As noted hereinabove, the electrodes can be disposed on an outer surface of the device (e.g., the base plate) such that the electrodes are in contact with the user's skin during use of the device. The electrodes can be used to generate biometric signals that can be processed by the biometric circuitry. In other embodiments, the biometric sensor 916 includes a PPG sensor. Notably, the PPG sensor includes at least one emitter and one detector electrically coupled to biometric circuitry. The emitter and detector can be configured to generate biometric signals that can be further processed by the biometric circuitry. Other EDA or bioimpedance sensor components can be utilized to generate biometric signals and can be further processed by biometric circuitry contained within the device.

The processor 902 is configured to obtain biometric signals from biometric sensors and can determine if the signal obtained from the biometric sensor satisfies a threshold signal strength. The processor 902 can also be configured to monitor, at least periodically, the signal strength of the biometric sensors on the device. For instance, in certain embodiments, the threshold signal strength can correspond to the magnitude or intensity that must be exceeded in order for optimal or desired operation of the biometric sensor 916. For instance, given that different types of biometric sensors (e.g., ECG and PPG) may be disposed on the device, the threshold signal strength for each can be different. However, in certain other embodiments, the threshold signal strength can define a range of signal strengths identified as proper, e.g., optimal or desired, for operation of the biometric sensor. Accordingly, the processor 902 can be configured to receive biometric signals from the biometric sensor 916 and can verify if the biometric signal falls within the acceptable threshold range of signals. Acceptable threshold signal strengths for various biometric sensors can be stored on the memory 904 and accessed by the processor 902. Stored acceptable threshold signal strengths may then also be used for defining at least one placement criterion fulfillment of which is to be detected when a placement of the wearable computing device on the user's extremity is to be adjusted.

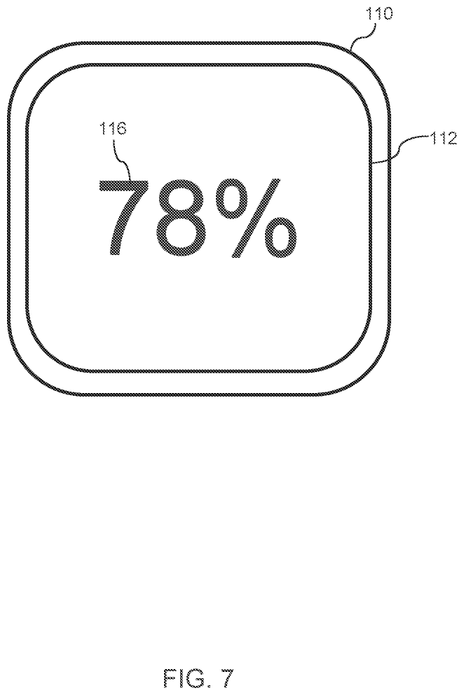

The processor 902 can be configured to trigger a control routine including one or more control actions for altering the signal strength of the biometric sensor, in the event that the biometric signal received is not within the threshold signal strength. For instance, upon determining that the biometric signal does not satisfy the threshold signal strength, the processor 902 can display a notification to the user. Such a notification is illustrated in FIG. 7. As shown, the notification 116 is displayed on the display 112 (or 906) of the housing 110. The notification can include an indicia indicative of the strength of the biometric signal to the user. As shown in FIG. 7, the biometric signal strength is shown as a percentage of the signal strength. In such embodiments, an acceptable threshold signal strength can be any range of percentages, such as those ranging from 80% to 100 %. Accordingly, the user will understand that display of an acceptable percentage means that the threshold signal strength is satisfied. In other embodiments, however, the notification can include a graphic notification indicative of biometric signal strength. In such embodiments, the notification can include color-coded emblems. For instance, display of a green emblem signifies that the threshold signal strength has been achieved, while display of either a yellow or red emblem signifies that it has not. Display of the yellow emblem, however, alerts the user that the threshold signal strength is not achieved, but that the biometric signal strength is stronger when the emblem is yellow as compared to when the red emblem is displayed. Any number of notifications indicative of the threshold signal strength can be displayed by the processor 902 as disclosed herein.

The processor 902 can be further configured to continually display the notification until the threshold signal strength is achieved. In such embodiments, the processor 902 can display a real-time value for the signal strength of the biometric signal. Referring back to FIG. 7, the percentage signal strength notification can be continually displayed on the display 112 (or 906) until the threshold signal strength is achieved. In embodiments, it is contemplated that upon receipt of the notification by the processor 902, the user may make one or more control actions including manually adjusting placement of the device on their extremity. In such embodiments, the processor 902 will continue to update the notification enabling the user to understand if modification of placement of the device on their extremity is strengthening or weakening the biometric signal. The processor 902 can display a notification once the threshold signal strength is achieved, for example, a green emblem or a percentage achieved. Such notification allows the user to understand that additional movement or positioning of the device is no longer necessary to alter placement of the wearable computing device 100 for improving the biosensor signal. Further, in certain embodiments, the notification can include a prompt to the user to modify placement of the device on the extremity to improve biosensor signal strength. The notification may also prompt the user to increase the pressure of the base plate against their skin to increase the biometric signal strength.

As noted, the processor 902 can send a notification to the user once threshold signal strength is achieved. The notification can be displayed on the display 112 (or 906) or the notification can be a sound alert, haptic alert, or combinations thereof.

In other embodiments, user input is required for the processor 902 to trigger the control routine for altering the signal strength of the biometric signal. For instance, in certain embodiments, the user can select an application via the display screen to initiate the control routine, including one or more control actions, by the processor 902. The user can also provide input to initiate the control routine via a user input, such as a button or dial located on the device.

In certain embodiments, the processor 902 can be configured to detect an on-extremity condition (i.e., to detect if the wearable computing device 100 is worn on an extremity of a user). For instance, when the device is powered but is not worn by the user, the processor 902 does not receive adequate signals from the biometric sensors and can determine that the device is in an off-extremity condition. However, upon the device being placed on the user's extremity the processor 902 receives stronger signals from the biometric sensors and can determine that the device is in an on-extremity condition. In such embodiments, the processor 902 can utilize a bioimpedance sensor (e.g., EDA) in order to determine the on-extremity condition. Detecting an on-extremity condition can trigger the processor 902 to initiate a routine to determine if biometric signals of one or more biometric sensors meet the threshold signal strength. If the biometric signal does not meet the threshold signal strength, the processor 902 can initiate the control routine for altering the signal strength of the biometric signal. In such embodiments, upon the user placing the device on their extremity (e.g., wrist) the processor 902 can facilitate proper placement of the device on the user's extremity such that the threshold signal strength is satisfied. In such a manner, the user can confirm proper placement and overall operation of the biometric sensors of the device upon initial placement of the device on their extremity.

As noted with respect to FIGS. 5A-5C, in certain embodiments, the base plate 122 of the device is coupled to the wearable computing device 100 via one or more actuators 500. In such embodiments, the processor 902 can be configured to facilitate translation of the actuators 500 in order to modify placement, in particular position, tightness and/or orientation of the base plate 122 on the user's extremity. For instance, upon detection by the processor 902 that threshold signal strength is not achieved, the processor 902 can initiate a control routine to modify the position of the base plate 122 in order to increase the biometric signal strength. For instance, the processor 902 can modify placement of the base plate 122 and then can assess whether such movement improves or worsens the biometric signal strength. In the event that such modification of the base plate 122 worsens the biometric signal strength, the processor 902 can initiate a process to modify placement of the base plate 122 in a different manner in order to improve biometric signal strength. The processor 902 can be configured to continue modification of the base plate 122 until threshold signal strength is achieved. In the event, however, that the processor 902 is able to achieve threshold signal strength, the processor 902 can then send a notification to the user, notifying the user of the low biometric signal strength and/or instructing the user to modify placement of the device in order to improve the biometric signal strength.

Still referring to example embodiments as shown in FIGS. 5A-5C, in certain embodiments a user input 114 can be used to translate the actuators 500 in order to move the base plate 122. For example, the user can utilize user input 114 to modify or adjust contact of the base plate 122 against the extremity of the user until a threshold signal strength is achieved. Optimal placement of the device can refer to a placement where the biometric signal strength meets the threshold signal strength as defined herein. In embodiments, the actuators 500 are coupled to a motor (not shown) disposed within the housing 110. The user input 114 can facilitate operation of the motor to move the actuators 500 and, thus, the base plate as desired by the user. In other embodiments, the actuators 500 are coupled to one or more racks and/or pinions disposed in the housing 110. The user can press, pull, and/or rotate the user input 114 to engage with the one or more racks or pinions disposed in the housing 110 in order to translate the actuators 500.

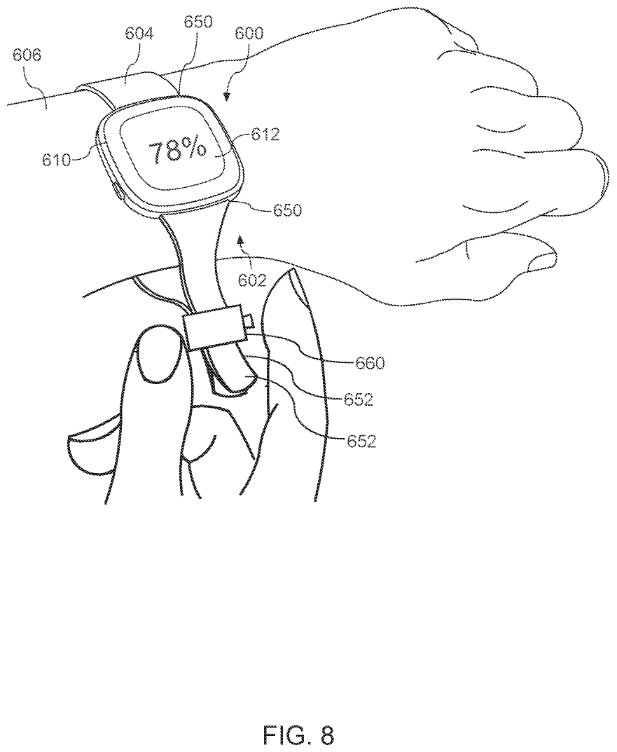

Now referring to FIG. 8, one or more bands 604 can be used to secure the device 600 to the extremity 606, such as the wrist 602 of the user. The device 600 includes a housing 610 and a display 612. The bands 604 are connected at a proximal end 650 to the housing 610 of the device and have a distal end 652 opposite from the proximal end 650. The proximal ends 650 of the bands 604 can be coupled to the housing 610 via an suitable mechanism, e.g., mechanically or adhesively. The proximal ends 650 of the bands 604 can include any suitable end attachment that allows for the bands 604 to be removed or replaced on the housing 610. Having the ability to remove the bands 604 from the housing 610 ensures that the user can replace the bands 604 as they desire for comfort or fashion. As shown in FIG. 8, a clasp 660 (e.g., a spring-loaded clasp) is coupled to the one or more bands 604. Notably, the distal ends 652 of the bands 604 can be placed through the clasp 660 and the clasp 660 can be translated up the length of the bands 604 towards the proximal ends 650 and the housing 610 in order to secure the device 600 to the user's wrist 602. Utilization of the bands 604 and clasp 660 can ensure that the device 600 fits in a snug manner on the user's wrist 602 as compared to other bands. For example, other bands including an indexed hole and mechanical prong, or clasp structure only allow for the user to select predetermined placement levels based on the spacing of the holes for receiving the prong or buckle. Such band configurations may not provide the level of tightness required for achieving a threshold signal strength as disclosed herein above.

The material for the bands 604 can also be selected to further facilitate a snug fit of the device 600. For instance, the bands 604 or at least portions of the bands 604, can be made from elastic materials including thermoplastic materials or rubbers. In certain embodiments, the bands can be made from thermoplastic urethanes. Fabrication of the bands 604 from an elastic material can facilitate tight fit of the device 600 on the user's wrist 602 and also expand or compress as the user moves their extremity 606 throughout the day. Different portions of the bands or end attachments can include elastic materials.

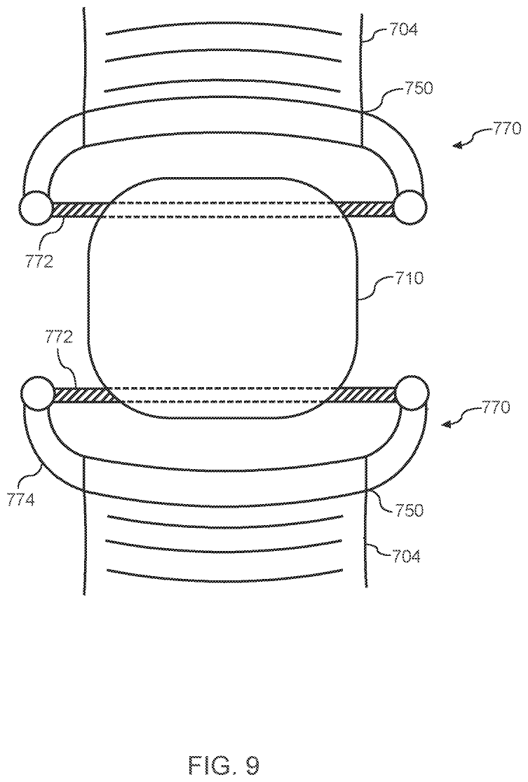

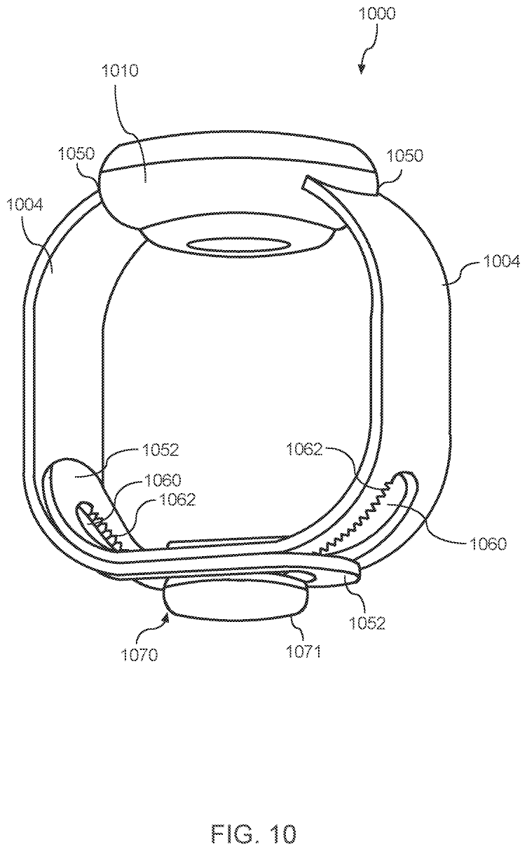

For example, as shown in FIG. 9, the proximal ends 750 of the bands 704 include end attachments 770. End attachments 770 include a prong 772 formed from an elastic material, as disclosed herein. The prong 772 is configured to be coupled to the housing 710 via any suitable manner. For instance, the prong 772 can be received in a channel (denoted by dashed line) located within the housing 710. The end attachment 770 can also include a fastener rod 774 coupled to both the prong 772 and the proximal end 750 of the band 704. The fastener rod 774 can be formed from the same material as the prong 772 or can be formed from a different material. For instance, the fastener rod 774 can include a metal material while the prong 772 includes an elastic material. In certain embodiments, the fastener rod 774 and prong 772 can be formed as a unitary structure. Notably, however, the fastener rod 774 and the prong 772 are separate, such that the prong 772 can be removed from the fastener rod 774 and replaced as desired by the user. In such embodiments, the prong 772 can help facilitate proper placement of the device on the wrist of the user. For instance, when the user tightens the bands 704, the elastic material of the prong 772 can expand or compress in order to adjust the pressure of the device 700 on the wrist of the user. For instance, should the user tighten the bands 704 too much, the prong 772 formed from elastic material can serve to reduce compressive forces exerted by the device 700 on the user's extremity. In such a manner, the prong 772 can further facilitate proper placement, including proper pressure of the device, on the user's skin.

FIG. 10 illustrates another embodiment of a wearable computing device 1000. One or more bands 1004 can be used to secure the wearable computing device 1000 to the user. The device 1000 includes a housing 1010. The bands 1004 are connected at a proximal end 1050 to the housing 1010 of the device and have a distal end 1052 opposite from the proximal end 1050. The proximal ends 1050 of the bands 1004 can be coupled to the housing 1010 via an suitable mechanism, e.g., mechanically or adhesively. The proximal ends 1050 of the bands 1004 can include any suitable end attachment that allows for the bands 1004 to be removed or replaced on the housing 1010. As shown in FIG. 10, apertures 1060 are disposed along the length of the distal end 1052 of the bands 1004. Each aperture 1060 is configured to mate with a ratchet mechanism 1070. For instance, the ratchet mechanism 1070 can include a housing 1071 having one or more gears or gear teeth disposed therein. The ratcheting mechanism 1070 can engage with teeth 1062 disposed along the perimeter of the apertures 1060 in order to tighten or loosen the bands 1004. For instance, the user can turn the ratchet mechanism 1070 to simultaneously tighten or loosen both of the bands 1004 the same distance. Such a band configuration allows for the user to equally tighten or loosen the bands with ease.

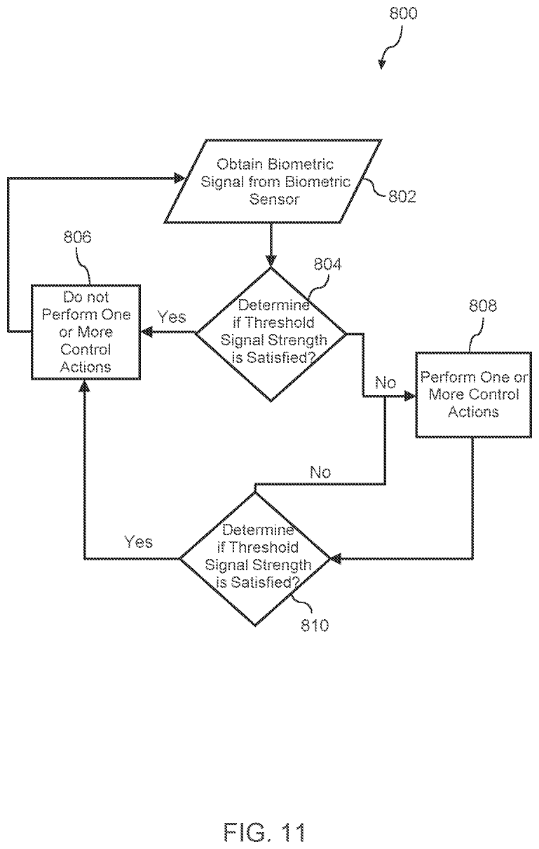

FIG. 11 is a flow chart of a method 800 for improving biometric sensor function on a wearable computing device. It should be understood that, for any process discussed herein, there can be additional, fewer, or alternative steps performed in similar or alternative orders, or in parallel, within the scope of the various embodiments.

At (802) a biometric signal is received from a biometric sensor. The biometric sensor may be formed in a wearable computing device and the wearable computing device can be configured to transmit the biometric signal from the biometric sensor to a processor.

At (804), the biometric signal strength is evaluated to determine if a threshold signal strength is satisfied. For example, the biometric signal is compared to a threshold signal strength to determine if the threshold signal strength is satisfied. The threshold signal strength can correspond to a certain magnitude, intensity, or quality that must be exceeded to facilitate optimal or desired operation of the biometric sensor. In other embodiments, the threshold signal strength can correspond to a desired range of magnitudes or intensity. In such embodiments, if the biometric signal falls within the range of magnitude or intensities, then the threshold signal strength is satisfied. A processor can be utilized to determine if the biometric signal satisfies the threshold signal strength.

At (806), if the threshold signal strength is satisfied at (804), then a control routine including one or more control actions is not initiated. However, if at (804) the threshold signal strength is not satisfied, then at (808) a control routine is initiated. The control routine can include one or more control actions that can be performed by the device, including a processor disposed therein, or by the user. The control actions can include those disclosed hereinabove. For example, suitable control actions can include displaying a notification to the user that the threshold signal strength is not satisfied. In other embodiments, the control action can include displaying a notification to the user to adjust placement of the device until the threshold signal strength is achieved. In other embodiments, the device itself can complete control actions, such as adjusting the base plate against the skin of the user to improve the biometric signal strength and/or to satisfy the threshold signal strength.

At (808) after performing one or more control actions, the biometric signal strength is evaluated to determine if the threshold signal strength is satisfied. The biometric signal strength can be determined as described above with reference to (804). If the biometric signal strength is satisfied, then at (806) no control actions are performed. However, if the threshold signal strength is not satisfied, then one or more control actions can be performed again per (808).

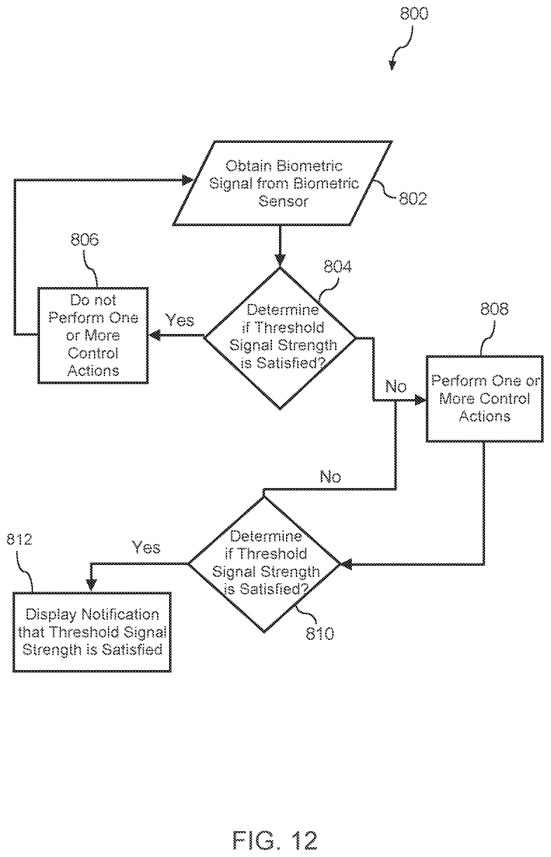

FIG. 12 is a flow chart of a method 800 for improving biometric sensor function on a wearable computing device. It should be understood that, for any process discussed herein, there can be additional, fewer, or alternative steps performed in similar or alternative orders, or in parallel, within the scope of the various embodiments. FIG. 12 is similar to the method shown in FIG. 11, however if at (810) the threshold signal strength is satisfied, then at (812) a notification is displayed indicating that the signal strength is satisfied. Again, the notification can include a notification on the display of the device, a sound alert, a haptic alert, or combinations thereof as described herein.

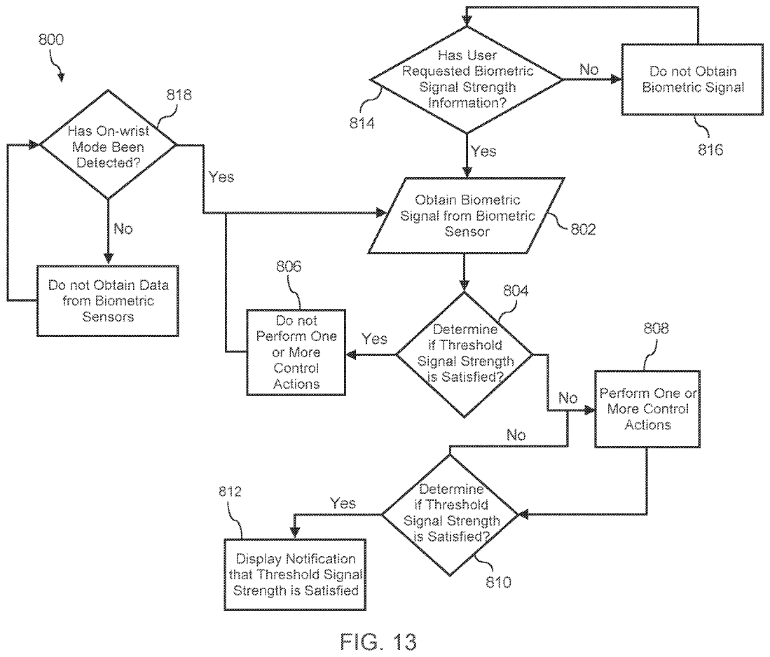

FIG. 13 is a flow chart of an example method 800 for improving biometric sensor function on a wearable computing device. It should be understood that, for any process discussed herein, there can be additional, fewer, or alternative steps performed in similar or alternative orders, or in parallel, within the scope of the various embodiments. FIG. 13 is similar to FIGS. 11-12, however, prior to obtaining the biometric signal at (802), at (814) user input is required. For example, if a user has provided input requesting information regarding the biometric signal strength, then at (802) the method proceeds with obtaining a biometric signal from the biometric sensor. If, however, the user has not requested information regarding the threshold signal strength, then at (816) no biometric signal is obtained. Indeed, in certain embodiments, method 800 as depicted in FIG. 13 may be suitable in situations where power utilized by the device needs to be conserved, for instance when the device has a low battery. In such instances, only input from the user can trigger the device to obtain and analyze the biometric signal strength. Such implementations can be automatically utilized when the device is operated in a low power mode. In such embodiments, the processor 902 is not continually obtaining biometric signals and evaluating them for threshold signal strength.

Similarly, at (818) the method can include detecting an on-extremity condition. If such an on-extremity condition is detected, then the method can include obtaining biometric signals from the biometric sensor per (802) and can proceed accordingly. If, however, an on-extremity condition is not detected, then at (818) no biometric signal is obtained.

While various embodiments have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the disclosure, but instead as merely intended to highlight alternative or additional features that may or may not be utilized in a particular embodiment of the disclosure. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor 902 or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

As used herein “threshold signal strength” can refer to either or both of the signal strength and/or the signal quality.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain embodiments of the disclosure are encompassed in the claim set listed below or presented in the future.

1-20. (canceled)

21. A wearable computing device comprising:
a housing;
a base plate coupled to the housing, the base plate defining a bottom surface of the housing, the base plate configured to contact an extremity of a user wearing the wearable computing device;
a display; and
a biometric sensor disposed on the base plate or within the housing, the biometric sensor configured to transmit a biometric signal; wherein
the wearable computing device is configured to:
obtain the biometric signal;
determine a signal strength of the biometric signal does not satisfy a threshold signal strength; and
responsive to determining the signal strength of the biometric signal does not satisfy the threshold signal strength, triggering a control routing comprising one or more control actions for altering the signal strength of the biometric signal.

22. The wearable computing device of claim 21, wherein the one or more control actions comprises displaying a notification prompting the user to manually adjust placement of the wearable computing device on the extremity of the user.

23. The wearable computing device of claim 21, wherein the one or more control actions comprises displaying a real-time value for the signal strength of the biometric signal.

24. The wearable computing device of claim 21, wherein the one or more control actions comprises providing a notification to the user once the threshold signal strength is achieved.

25. The wearable computing device of claim 21, wherein the one or more control actions comprises continually displaying a notification on the display until the threshold signal strength is achieved.

26. The wearable computing device of claim 21, wherein the one or more control actions comprises providing a notification to the user when the threshold signal strength is not satisfied.

27. The wearable computing device of claim 21, wherein the wearable computing device is configured to detect an on-extremity condition.

28. The wearable computing device of claim 21, wherein a user input is required for the wearable computing device to perform the one or more control actions.

29. The wearable computing device of claim 21, wherein the one or more control actions comprises utilizing one or more actuators to adjust the base plate against the extremity of the user.

30. The wearable computing device of claim 29, wherein the one or more actuators are configured to move the base plate towards the extremity of the user in a first direction and away from the extremity of the user in a second direction that is opposite from the first direction.

31. The wearable computing device of claim 21, wherein the one or more control actions comprises utilizing an input device disposed on the housing configured to control one or more actuators to adjust contact of the base plate against the extremity of the user until the threshold signal strength is achieved.

32. The wearable computing device of claim 21, wherein the base plate is spring-mounted to the housing.

33. The wearable computing device of claim 21, wherein the biometric sensor comprises an electrocardiogram (ECG) sensor, a photoplethysmography (PPG) sensor, a bioimpedance sensor, an electrodermal activity sensor (EDA), or combinations thereof.

34. The wearable computing device of claim 21, further comprising:
one or more bands configured to attach the wearable computing device to the extremity of the user.

35. The wearable computing device of claim 34, comprising a spring-loaded clasp coupled to the one or more bands for adjusting a tightness of the one or more bands against the extremity of the user.

36. The wearable computing device of claim 34, wherein the one or more bands comprise one or more end attachments configured to be coupled to the housing, wherein the one or more end attachments comprises an elastic prong configured to be received in a groove in the housing.

37. The wearable computing device of claim 34, wherein each of the one or more bands comprise an aperture configured to mate with a ratchet mechanism, the ratchet mechanism configured to tighten or loosen the one or more bands.

38. A method for improving biometric sensor function, comprising:
obtaining a biometric signal from a biometric sensor disposed on a base plate, the base plate adapted to contact an extremity of a user while being worn by the user;
determining a signal strength from the biometric sensor does not satisfy a threshold signal strength; and
responsive to determining the signal strength does not satisfy the threshold signal strength, triggering a control routine comprising one or more control actions for altering the signal strength of the biometric signal.

39. The method of claim 38, wherein performing the one or more control actions comprises continuously displaying a notification on the display until the signal strength of the biometric signal from the biometric sensor satisfies the threshold signal strength.

40. The method of claim 38, wherein performing one or more control actions comprises adjusting one or more actuators configured to adjust the base plate in a first direction against the extremity of the user or in a second direction away from the extremity of the user until the threshold signal strength is achieved.
